(19) United States
(12) Patent Application Publication
(10) Pub. No.: US 2005/0133037 A1
(43) Pub. Date: Jun. 23, 2005

(54) Laryngeal mask assemblies

(75) Inventor: Jeremy Colin Russell, Hythe (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 11/012,827

(22) Filed: Dec. 16, 2004

(30) Foreign Application Priority Data
Dec. 18, 2003 (GB) 0329298.4

(51) Int. Cl. A61M 16/00

(52) U.S. Cl. 128/207.15; 128/200.26

(57) ABSTRACT

A laryngeal mask assembly has a sealing cuff supported on a mount at the patient end of a tube. The tube has two portions of substantially equal length, the patient end portion being unreinforced and the machine end portion being reinforced by a helical metal wire and being more flexible than the patient end.

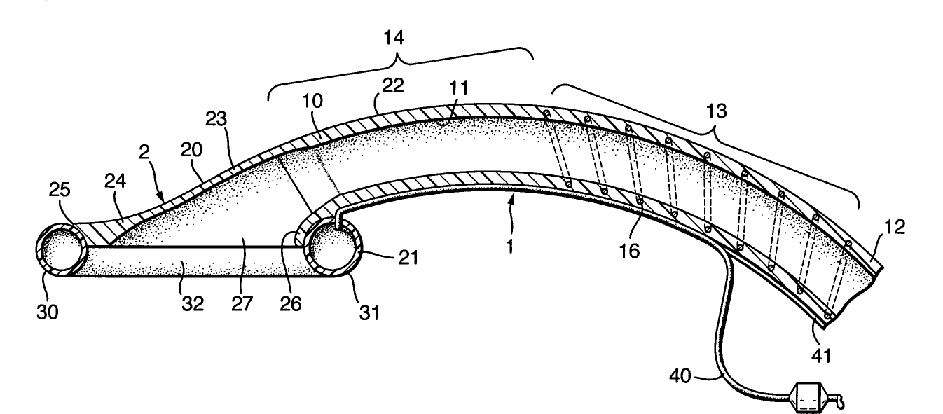

### BACKGROUND OF THE INVENTION

This invention relates to laryngeal mask assemblies and their manufacture

It is common practice to use an airway known as a laryngeal mask for administering anaesthetic and ventilation gases to a patient. These airways comprise a tube with an inflatable mask or cuff at one end, the tube being inserted in the patient's mouth so that one end is located in the hypopharynx and so that the mask forms a seal in this region with the surrounding tissue. Laryngeal masks are described in, for example, U.S. Pat. No. 5,355,879, U.S. Pat. No. 5,305,743, U.S. Pat. No. 5,297,547, U.S. Pat. No. 5,282,464, GB2267034, U.S. Pat. No. 5,249,571, U.S. Pat. No. 5,241,956, U.S. Pat. No. 5,303,697, GB2249959, GB2111394, EP448878, U.S. Pat. No. 4,995,388, GB2205499, GB2128561, GB2298797, GB2334215, GB2337020, PCT/GB00/03044, PCT/GB00/03045, GB0002805, GB0020274 and GB2371990. Laryngeal masks usually comprise a curved, extruded shaft, a separate mount member joined at the patient end of the tube and an inflatable cuff attached to the mount member. The shaft may be reinforced along its length with a helical reinforcing element such as a wire.

Laryngeal masks have several advantages over endotracheal tubes, which are longer and seal with the trachea below the vocal folds. Reinforced laryngeal masks have some advantages in that the shaft can be made flexible, so that it can be bent sharply where it emerges from the patient's mouth, without the risk of kinking. Also, the risk of the shaft being occluded by pressure from the teeth is reduced. The increased flexibility of the shaft, however, has disadvantages in that it can make placement and retention of the patient end of the mask more difficult.

### BRIEF SUMMARY OF THE PRESENT INVENTION

It is an object of the present invention to provide an alternative laryngeal mask assembly.

According to the present invention there is provided a laryngeal mask assembly comprising a tube, a mount at the patient end of the tube, and an annular sealing cuff extending around the patient end of the mount, the tube having a first portion that is helically reinforced and a second portion that is unreinforced, the first portion being arranged in use to extend through lips of the patient, and the second portion being arranged to extend into the pharyngeal region.

The first and second portions are preferably of substantially equal length. The reinforced portion may be formed separately of the unreinforced portion or be formed integrally of the unreinforced portion. The reinforced portion is preferably more flexible than the unreinforced portion. The first portion is preferably reinforced by a helically wound metal wire. The mount may be formed integrally at the patient end of the tube.

A laryngeal mask assembly according to the present invention will now be described, by way of example, with reference to the accompanying drawing.

### BRIEF DESCRIPTION OF THE DRAWING

The drawing is a side elevation view of the assembly.

### DETAILED DESCRIPTION OF THE PREFERRED EMBODIMENT

The laryngeal mask assembly comprises a tube 1 and a mask formation 2 at the patient end 10 of the tube.

The tube 1 is of a bendable plastics material, such as PVC and is curved along its length. A bore 11 extends along the tube from its patient end 10 to its rear, machine end 12. The tube 1 is divided along its length into two portions of substantially equal length, namely a reinforced portion 13 and an unreinforced portion 14. The reinforced portion 13 is at the machine end of the assembly and, in use, extends out of the mouth between the teeth and lips of the patient. The unreinforced portion 14 is at the patient, mask end of the assembly and, in use, extends within the oral cavity and into the pharyngeal region. The reinforced portion 13 is reinforced by a helical reinforcement element such as a helical metal wire 16 although it could be of a stiff plastics material. The reinforced portion 13 is preferably more flexible than the unreinforced portion 14. This could be achieved by a reduced wall thickness or by a softer grade of plastics in the reinforced portion.

There are various ways in which the tube 1 could be formed. The reinforced and unreinforced portions could be made separately and subsequently joined end-to-end with each other. Alternatively, however, the two portions could be formed integrally with one another such as by overmoulding onto the reinforcement element.

The mask 2 comprises a mount 20 and an inflatable sealing cuff 21. The mount 20 is of a relatively stiff plastics material and is of generally shoe shape. The mount 20 is moulded integrally at the forward end of the tube 1, such as by injection moulding, to form an integral, single piece 22. The mount 20 tapers outwardly from its machine end 23 to its patient end 24, which is inclined to the axis of the machine end at an angle of about 25° so that the patient end of the mount has an oval shape with its forward end 25 being more pointed than its rear end 26. The patient end 24 of the mount 20 is inclined to face towards the inner side of the curve of the tube 1. Internally, the mount 20 has a cavity 27 that increases in cross-sectional area along its length, from the machine end.

The cuff 21 is tubular and of a thin flexible plastics material. The cuff 21 is formed into an annulus of the same shape as the patient end 24 of the mount 20 so that it is oval with its forwardly-directed end 30 being more pointed than its rearwardly-directed end 31. The cuff 21 encloses a central region 32 of the same shape as the patient end 24 of the mount 20. The cuff 21 is attached around the patient end 24 of the mount 20 such as by means of an adhesive. The cuff 21 is inflated and deflated by means of an inflation line 40 which is provided by a separate small-bore tube communicating with the interior of the cuff and extending rearwardly along a groove 41 in the outside of the tube. When inflated in position in a patient, the cuff 21 expands to contact patient tissue in the region of the hypopharnyx.

The reinforced portion 13 of the tube 1 projecting from the mouth can be bent down or to one side to keep the region around the mouth clear and without the risk of kinking the tube. Because the patient end of the tube is unreinforced the mount can be made integrally with the patient end of the tube, thereby enabling the region located within the larynx to be smooth, with no abrupt transition. The assembly can be easier to place because the distal, patient end can be stiffer than the proximal machine end. The cost of the assembly is can be less than that of a tube reinforced along its entire length because of the reduced length of reinforced tube, which is more expensive than unreinforced tubing.

1. A laryngeal mask assembly of the kind comprising: a tube having a patient end and a machine end; a mount at said patient end of said tube, said mount having a patient end and a machine end; and an annular sealing cuff extending around said patient end of said mount, the improvement wherein said tube has a first portion that is helically reinforced and a second portion that is unreinforced, wherein said first portion is arranged in use to extend through lips of a patient, and wherein said second portion is arranged to extend into a pharyngeal region of the patient.

2. A laryngeal mask assembly according to claim 1, wherein said first and second portions are of substantially equal length.

3. A laryngeal mask assembly according to claim 1, wherein said first portion is formed separately of said second portion.

4. A laryngeal mask assembly according to claim 1, wherein said first portion is formed integrally of said second portion.

5. A laryngeal mask assembly according to claim 1, wherein said first portion is more flexible than said second portion.

6. A laryngeal mask assembly according to claim 1, wherein the first portion is reinforced by a helically wound metal wire.

7. A laryngeal mask assembly according to claim 1, wherein said mount is formed integrally at said patient end of said tube.
